(19) United States
(12) Patent Application Publication
(10) Pub. No.: US 2021/0031015 A1
(43) Pub. Date: Feb. 4, 2021

(54) PROTECTED PORTS AND CATHETERS

(71) Applicant: Nikola Dobrilovic, Boston, MA (US)

(72) Inventor: Nikola Dobrilovic, Boston, MA (US)

(21) Appl. No.: 16/524,625

(22) Filed: Jul. 29, 2019

(51) Int. Cl. A61M 39/02

(52) U.S. Cl. CPC A61M 39/0208; A61M 25/0097

(57) ABSTRACT

A protected port or catheter generally includes a cannula having at least one lumen and at least one access point in fluid communication with the at least one lumen. An access control mechanism controls fluid communication between the access point and the at least one lumen and is operable between closed and open positions. The access control mechanism may include a valve, a door, or a lockable cap. An actuator may be included for operating the access control mechanism between open and closed states. The actuator may be selectively operated by a control circuit. A power source may be provided to power the control circuit and actuator, if included.

### BACKGROUND OF THE INVENTION

### 1. Field of the Invention

The present patent document relates generally to ports for delivering medications and other intravenous fluids to a patient, and more particularly to a protected port or catheter that prevents tampering or unauthorized injections therethrough.

### 2. Background of the Related Art

Intravenous and intravascular catheters, such as short and midline peripheral catheters, peripherally inserted central catheters (“PICC” or “PICC line”), tunneled central venous catheters (“CVC”), percutaneous non-tunneled catheters, include a tubular cannula which may include one or more lumens, for insertion into a vein or artery, and a tail with one or more caps with ports, corresponding to each lumen, for delivery of medications and fluids into the cannula and to the patient. A manual clamp may be included on the tails near the caps to close or restrict medication and fluid transfer from the ports on the caps. Intravascular catheters may remain in situ for extended periods of time, such as for days, months or even a year.

Prior art implantable ports are medical devices that consist of two components. The first is a thin, soft, plastic tube called a catheter that is typically inserted (tunneled) under the skin of the patient and courses over into a large vein. The catheter tubing connects to the second component called a port reservoir that is implanted under the skin. The port reservoir will show as a small bump underneath the patient's skin, which can be felt but is not directly visible on the outside of the body. To use the port, a medical professional will pass a special type of needle, such as a Huber needle, through the skin into the port reservoir allowing medicines to be given into the vein or blood to be taken from the vein.

Implanted ports function in a manner similar to regular intravenous or intravascular catheters and can be used to inject medications or other intravenous fluids such as chemotherapy, blood transfusions, nutritional liquids, or antibiotics. Blood can be drawn from the body through the port when it is needed for testing. Having the port implanted makes it possible for the patient to have their treatment without the need to frequently/repeatedly obtain intravenous access each time. A port may also be very useful if medical professionals find it difficult to get needles into the patient's veins, or if the walls of the patient's veins have been hardened by previous treatment. Often ports are placed so that a patient can undergo a “long-term” therapy as an outpatient. This allows patients to be discharged home. They can then receive medical treatments at home by a visiting medical provider or alternatively come to a medical facility as needed for infusion of medicine and then return home.

However, patients that are intravenous drug abusers have the potential to cause serious harm to themselves if they are provided with a long-term catheter or an implantable port. A drug abusing patient, if not monitored carefully, may use the port to inject narcotics directly into their veins, possibly killing themselves by overdosing or by causing an infection. Medical professionals faced with this situation are confronted with the dilemma of sending the patient home with the risk the port could be abused; or using costly inpatient treatment, for what would otherwise qualify as an outpatient treatment for any other patient. In the medical community, it is uniformly accepted that known IV drug users pose a tremendous safety risk and liability (medicolegal). They are, therefore, often kept in the hospital under inpatient status until medical therapy has been completed. It is not uncommon for treatments to last eight weeks. This creates a large inefficiency and cost burden on medical professionals and facilities.

Therefore, there is a need in the medical industry for a catheter or port that is safeguarded from unauthorized use.

### SUMMARY OF THE INVENTION

The present invention solves the problems of the prior art by providing a protected port or catheter that prevents tampering from the patient or other unauthorized person. The protected port or catheter generally includes a cannula having at least one lumen and at least one access point in fluid communication with at least one lumen. An access control mechanism controls fluid communication between the access point and at least one lumen and is operable between closed and open positions via an actuator, which may be selectively operated by a control circuit. The access control mechanism may include a valve, a door, or a lockable cap. A power source may be provided to power the control circuit and actuator.

### BRIEF DESCRIPTION OF THE DRAWINGS

Various aspects of at least one embodiment are discussed below with reference to the accompanying figures, which are not necessarily drawn to scale, emphasis instead being placed upon illustrating the principles disclosed herein. The figures are included to provide an illustration and a further understanding of the various aspects and embodiments, and are incorporated in and constitute a part of this specification, but are not intended as a definition of the limits of any particular embodiment. The figures, together with the remainder of the specification, serve only to explain principles and operations of the described and claimed aspects and embodiments, but are not to be construed as limiting embodiments. In the figures, each identical or nearly identical component that is illustrated in various figures is represented by a like numeral. For purposes of clarity, not every component may be labeled in every figure.

FIG. 1 is a view of an embodiment of an implantable protected port shown implanted inside a patient with a cannula inserted in a blood vessel;

FIG. 2 is a cross-section of a first embodiment of a port having a port door controlling access to a septum;

FIG. 3 is a top view of a first embodiment of a port having a port door controlling access to a septum;

FIG. 4A is a partial cross-section through line 4A-4A of FIG. 2 of a first embodiment of a port having a port door controlling access to a septum with the port door in a closed position;

FIG. 4B is a partial cross-section of a first embodiment of a port having a port door controlling access to a septum with the port door in an open position;

FIG. 5 is a diagram of an exemplary control circuit for a protected port or catheter;

FIG. 6 is a flowchart of an exemplary method of communicating with an access point of an embodiment of a protected port or catheter made in accordance herewith;

FIG. 7A is a side cross-section view of a second embodiment of a protected catheter having a valve controlling access to a lumen of a catheter;

FIG. 7B is a diagram view of a second embodiment of a protected catheter having a valve controlling access to a lumen of a catheter;

FIG. 8A is a perspective view of a third embodiment of a protected catheter configured as a long-term use intravascular catheter with a valve control mechanism;

FIG. 8B is a partial schematic diagram of a third embodiment of the protected catheter of FIG. 8A;

FIG. 9A is a perspective view of a fourth embodiment of the protected catheter configured as a long-term use intravascular catheter with a locking mechanism in a closed position;

FIG. 9B is a partial exploded view of a fourth embodiment of the protected catheter configured as a long-term use intravascular catheter with a locking mechanism in an open position, showing removal of a locking cap;

FIG. 9C is a is a close-up view of a fourth embodiment of the protected catheter configured as a long-term use intravascular catheter with a locking mechanism in an open position;

FIG. 10A is a perspective view of a fifth embodiment with a protected port configured as a long-term use intravascular catheter with a leaf shutter mechanism;

FIG. 10B is a close-up, perspective view of a fifth embodiment with a protected port with a leaf shutter mechanism in a closed position; and

FIG. 10C is a close-up, perspective view of a fifth embodiment with a protected port with a leaf shutter mechanism in an open position.

### DESCRIPTION OF THE PREFERRED EMBODIMENT

The examples of the apparatus discussed herein are not limited in application to the details of construction and the arrangement of components set forth in the following description or illustrated in the accompanying drawings. It will be understood to one of skill in the art that the apparatus is capable of implementation in other embodiments and of being practiced or carried out in various ways. Examples of specific embodiments are provided herein for illustrative purposes only and are not intended to be limiting. Also, the phraseology and terminology used herein is for the purpose of description and should not be regarded as limiting. Any references to examples, embodiments, components, elements or acts of the apparatus herein referred to in the singular may also embrace embodiments including a plurality, and any references in plural to any embodiment, component, element or act herein may also embrace embodiments including only a singularity (or unitary structure). References in the singular or plural form are not intended to limit the presently disclosed apparatus, its components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

As described in greater detail below, embodiments of the protected port or catheter 10, 100, 200, 300, 400 may be configured for subcutaneous or external use. The protected port or catheter generally includes an access point, a control circuit having a communications module, an actuator, and an access control mechanism controlling access to the access point. The protected port or catheter may be powered with batteries or include a wireless power transmission method, such as electric, magnetic or electromagnetic induction. Communication with the protected port or catheter may be through magnetic or wireless communication method. The control circuit on the protected port or catheter, receives a request to access the access point through a communications module. The control circuit verifies the authenticity of the request, and, via the actuator, opens the access control mechanism. The control circuit may also receive, through the communications module, a request to close the access point. After verifying the authenticity of the request, the control circuit operates the actuator to close the access control mechanism.

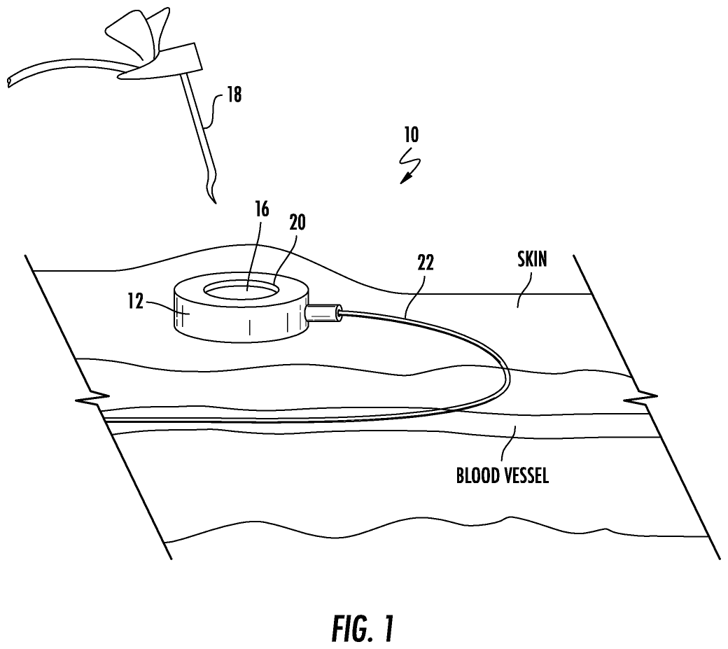

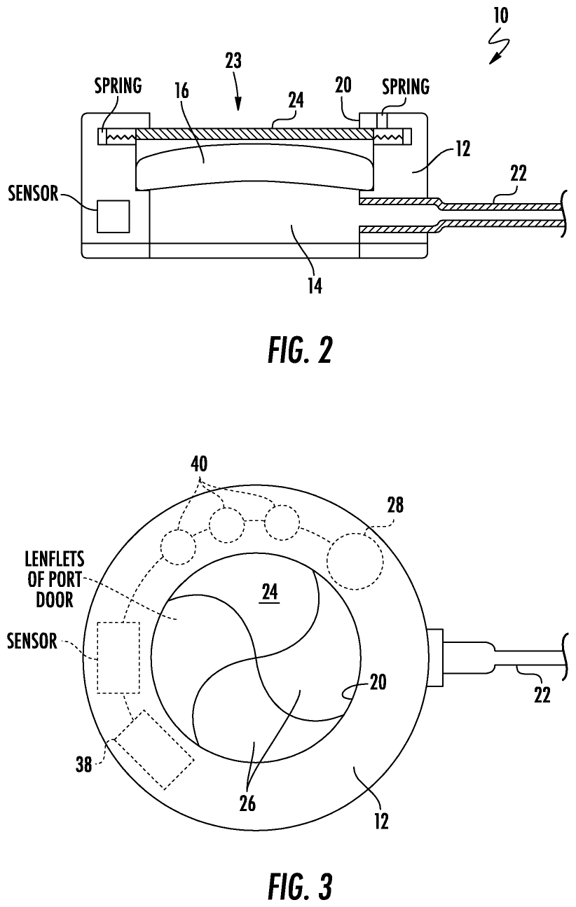

Referring to FIGS. 1-4B, a first embodiment of a protected port or catheter is shown generally at 10. In the first embodiment, a protected port or catheter is configured for subcutaneous implantation as an implantable port. The first embodiment includes a two-part housing 12 with a reservoir 14 contained inside. The housing 12 may be formed from plastic or metal as is known in the art. The reservoir 14 includes an access point, such as a septum 16 made from an elastic material, such as a silicone resin, for receiving a needle of a syringe or Huber needle 18. The housing 12 includes an opening 20, permitting access to the septum 16. A cannula 22 having a single lumen is connected to the housing 12 and is in fluid connection with the reservoir 14. The cannula 22 may be detachable from the housing 12. The housing 12 further includes an access control mechanism 23, such as a port door 24, configured to close the opening 20, thereby selectively preventing access to the septum 16.

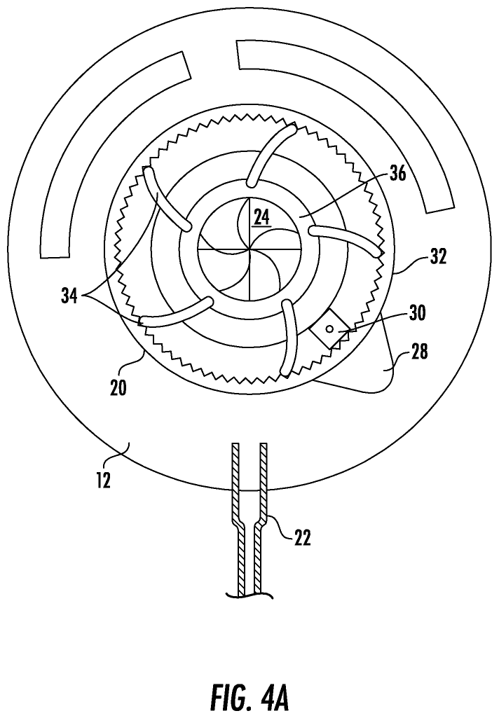

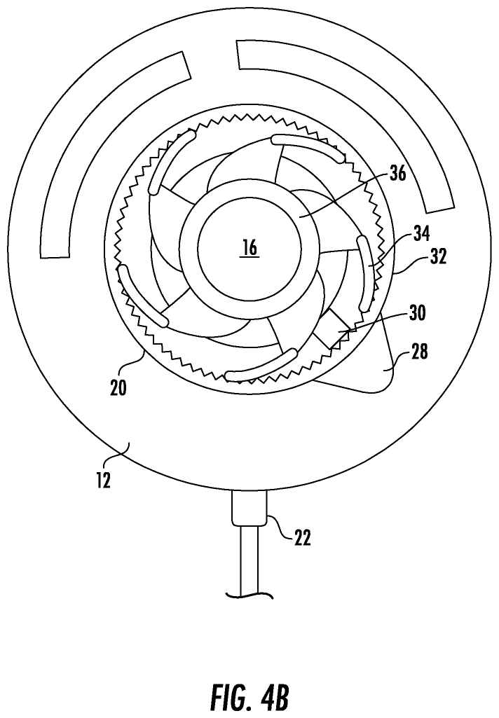

The port door 24 may be formed in a number of configurations, such as an iris having one or more leaflets 26 that slide or pivot between an open state and a closed state. In the closed state, the leaflets 26 of the port door 24 slide or pivot into position, obscuring and blocking access to the septum 16, thereby preventing a needle 18 from being inserted into the reservoir 14. In the open state, the leaflets 26, slide or pivot out of the way, allowing access to the septum 16. As show in FIGS. 4A and 4B, an actuator 28 drives a first gear 30, which drives an outer second gear 32. The second gear 32 pulls a number of arms 34, each pivotally attached to a leaflet 26. The leaflets 26 are pivotally retained in a bracket 36 at the opening 20 in the housing 12. Rotation in one direction opens the leaflets 26, and closes the leaflets 26 in the reverse direction. In another embodiment, the port door 24 may be spring-biased closed, with the door mechanism configured to open the port door 24 and, after treatment, release the port door 24, allowing the spring to return the port door 24 to a closed position. In another embodiment, the port door 24 may be spring-biased to the open position, with the door mechanism closing the port door 24.

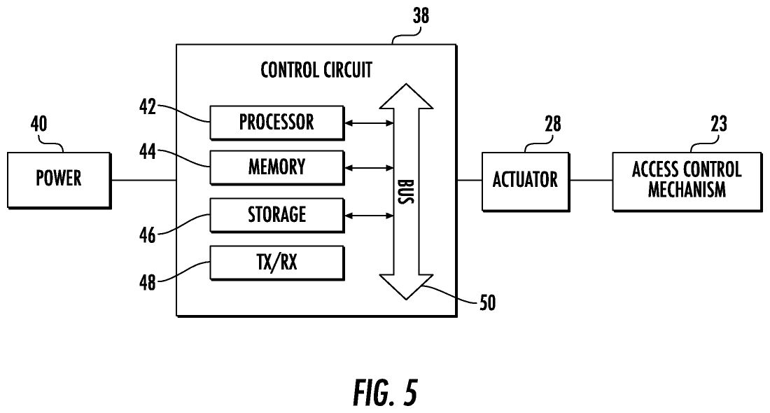

Referring to FIG. 5, an exemplary diagram for a protected port or catheter is shown generally. Use of the access point, such as the septum 16 is controlled by a control circuit 38, which operates the actuator 28 to drive an access control mechanism 23 between the open and closed positions. Power to the circuit 38 and actuator 28 is supplied via a power supply 40. The control circuit 38 may include a processor 42, a memory 44, a storage 46, and a communications module 48, interconnected via a bus 50. The communications module 48 may be configured to receive and transmit via a magnetic or wireless communication method, such as near-field magnetic induction or use of a radio frequency protocol, such as Zigbee, Bluetooth low energy, Wi-Fi, IEEE 802.15.4, Z-Wave, or a proprietary protocol, by way of example and not limitation. The power supply 40 may include a battery that is rechargeable via induction and/or a wireless power receiver that is configured to receive pulsed or modulated electric, magnetic or electromagnetic waves and to convert into power.

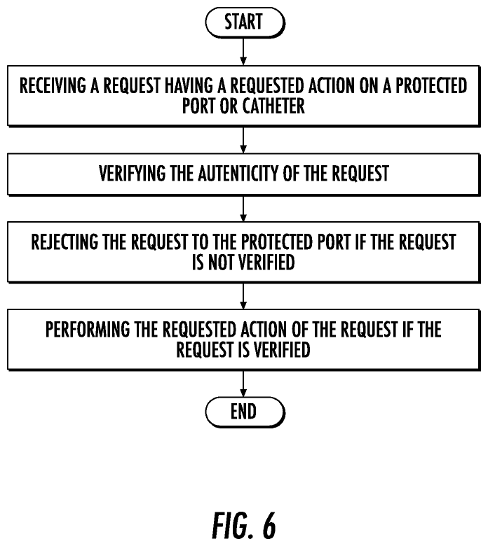

Referring to FIG. 6, a method of sending a request to a protected port or catheter is shown generally. In a first step, the control circuit 38 receives, via the communications module 48, a request having a requested action, such as to open or close the access point on the protected port or catheter, for instance. In a second step, the control circuit 38, with the processor 42, verifies the authenticity of the request. In some embodiments, the request is verified by use of a secret code or PIN, which is matched against a code stored in a storage 46 of the control circuit 38. In other embodiments, the authenticity of the request may be verified by the mode of communication, such as the use of a proprietary communication protocol or the use of certain frequencies, proximity, signal strength or pulse modulation in the wireless communication. In other embodiments, a lookup table containing device identifiers of authorized devices is maintained on the storage 46. If the request is not verified as being authentic, the request, via the processor 42, is rejected in a fourth step. If the request is verified as being authentic, the control circuit 38, via the processor 42, performs the requested action, e.g. opens the access point, in a fifth step. Other requested actions may include transmitting diagnostics, usage history, changing the secret code or PIN, updating firmware in storage of the control circuit, performing a system reset, and performing a system shutdown, for example and not by way of limitation.

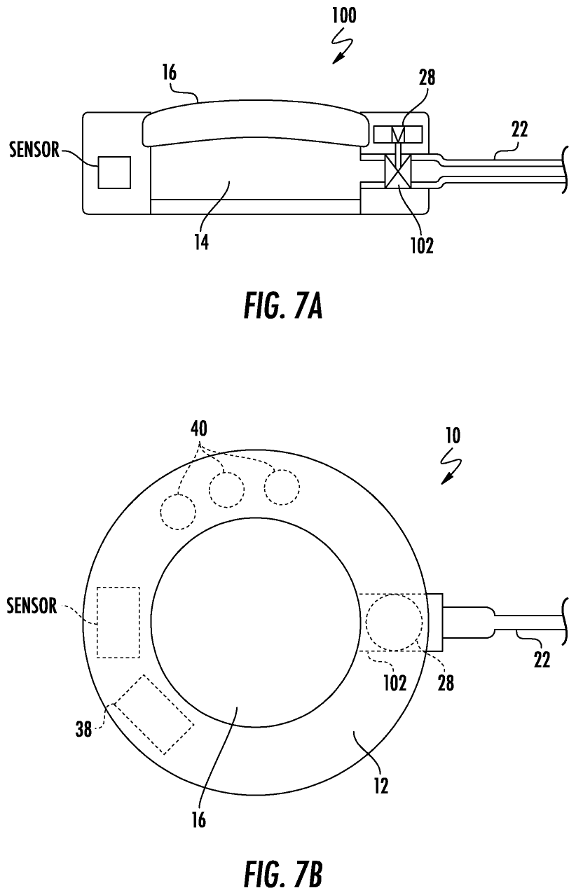

Referring to FIGS. 7A and 7B, a second embodiment of a protected port is shown generally at 100. In the second embodiment 100, a valve 102 controls access to the lumen of the cannula 22. Instead of a door controlling access to the septum 16, a valve 102 controlling the lumen of the cannula 22 may be used. Access to the septum 16 is unimpeded. Because the valve 102 may be selectively opened and closed, wirelessly, the port 100 may not be used to inject unauthorized drugs into the patient. In such an embodiment, the outer surface of the lumen must necessarily be reinforced or armored in a manner that prevents inserting a needle for injection directly into the lumen to circumvent the protective control valve.

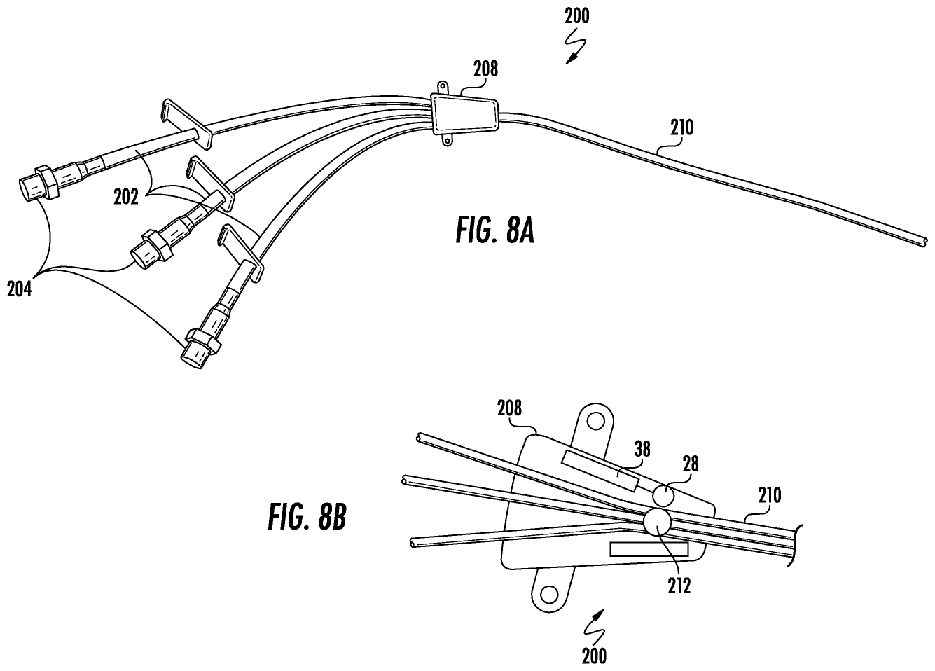

Referring to FIG. 8A and 8B, a third embodiment of the protected port or catheter is shown generally at 200. The third embodiment 200 of the protected port or catheter is shown generally configured as a triple-lumen catheter for intravenous use. The third embodiment 200 includes three tails 202 with caps 204, one for each lumen 206 of the catheter, that connect to a body or hub 208. A cannula 210, containing the lumens 206, extends from the hub 208 and is configured for insertion into the body of a patient. Inside the hub 208 is a control circuit 38 configured to operate an actuator 28 to manipulate a valve 212 between open and closed positions on the cannula 210, which simultaneously controls all three lumens 206. Multiple valves 212 may be included to control use of individual tails 202 of the catheter, selectively. A portion or all of the cannula 210 may include a needle-resistant outer sheath, to prevent insertion of a needle into the cannula 210 to circumvent the valve 212. The outer sheath may be integral to the cannula 212. Optionally, the tails 204 of the catheter may also include a needle-resistant outer sheath for added anti-tamper protection. Each tail 202 may further include a shutoff 214, configured to crimp the tail 202 closed, as is known in the art. In such an embodiment, the outer surface of the distal portion of the lumen and even the subcutaneous portion must necessarily be reinforced or armored in a manner that prevents inserting a needle for injection directly into the lumen to circumvent the protective control valve.

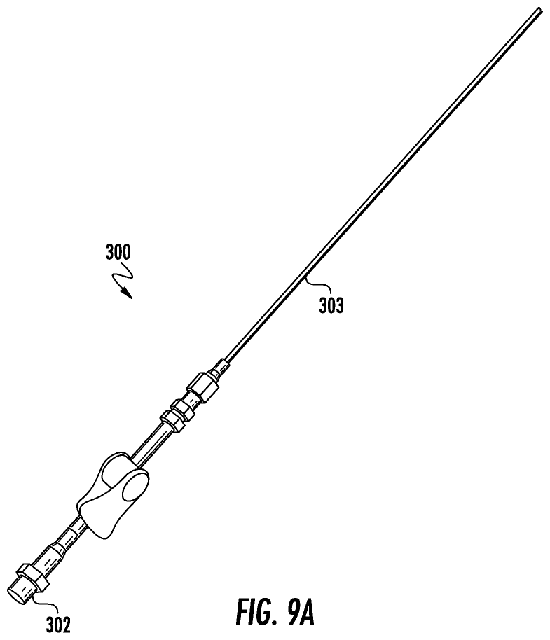

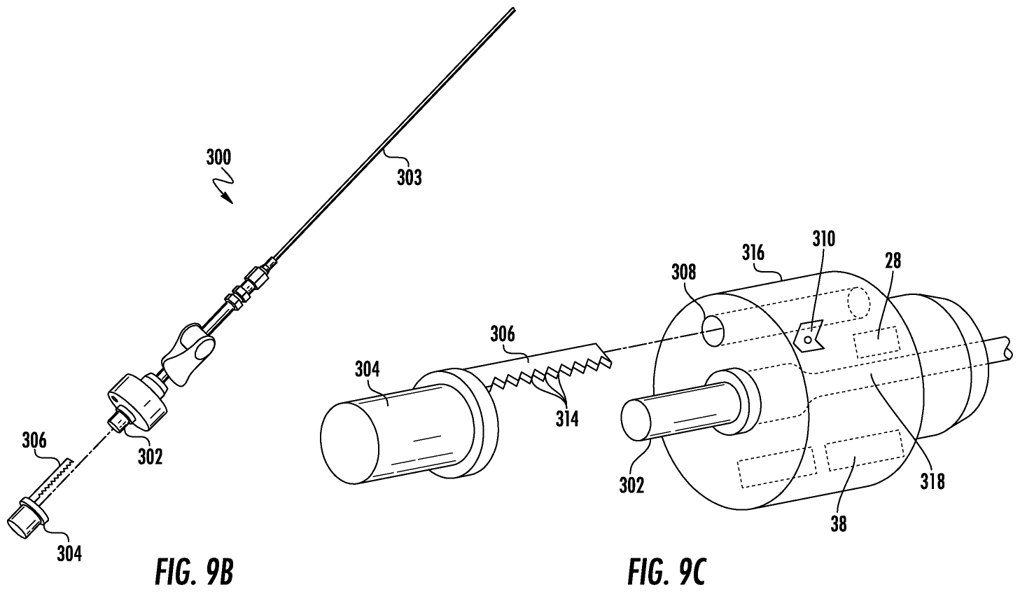

Referring to FIGS. 9A-9C, a fourth embodiment of the protected port or catheter is shown generally at 300. In the fourth embodiment 300, includes a single lumen 303 having an access point comprising a cap 302 with a connector configured for receiving a needle of a syringe or an intravenous line connected thereto. The access control mechanism comprises cover 304 with a toothed shaft 306 received into an opening 308 of a locking mechanism 312 with a spring-biased pawl 310 of a body 316. The cover 304 is configured to fit over the cap 302 and prevent use of the cap 302. The teeth 314 on the shaft 306 have a sawtooth pattern and form a one-way ratchet against a spring-biased pawl 310 in the locking mechanism 312. The pawl 310 in the locking mechanism 312 cooperates with the teeth 314 on the toothed shaft 306, allowing the shaft 306 to slip by when being inserted, but preventing removal as the pawl 310 stops against the sawtooth pattern of the teeth 314. The actuator 28 selectively retracts the pawl 310 on the locking mechanism 312 to release the toothed shaft 306, and thereby permitting the cover 304 to be removed. The actuator 28 may automatically release the pawl 310 after a predetermined amount of time has lapsed. Alternatively, the user may send a request to close the port, which causes the control circuit 38 and actuator 28 to release the pawl 310. To re-secure the catheter 300, the user then inserts the toothed shaft 306 of the cap 304 into the opening 308 on the locking mechanism 312, and presses it closed. The lumen 302 of the catheter 300 may include a needle-resistant outer sheath, to prevent insertion of a needle into the lumen 303 to circumvent the cover 304 over the cap 302. The outer sheath may be integrally formed with the lumen 303 of the catheter 300. The body 316 of the protected port may be integrally formed with the cap 302 of the catheter 300. Alternatively, the body 316, may include a central bore 318 sized and dimensioned to received the lumen 303 and cap 302, thus allowing the protected port to be reused.

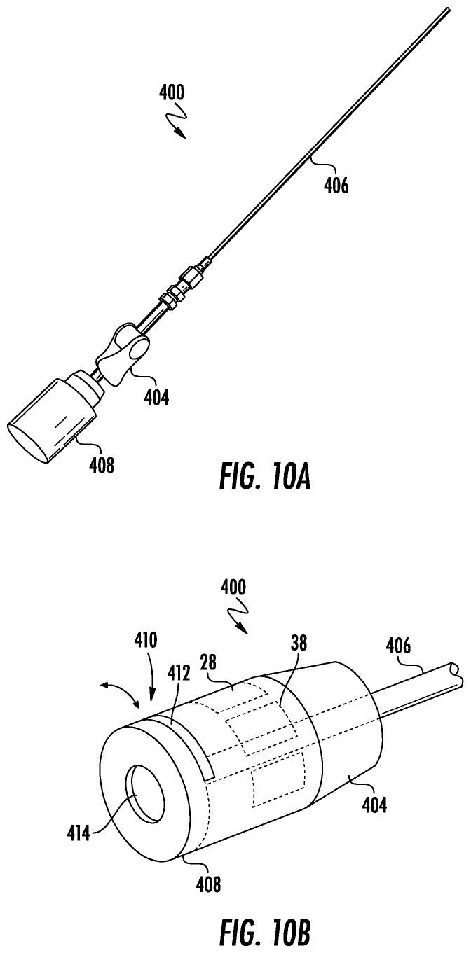

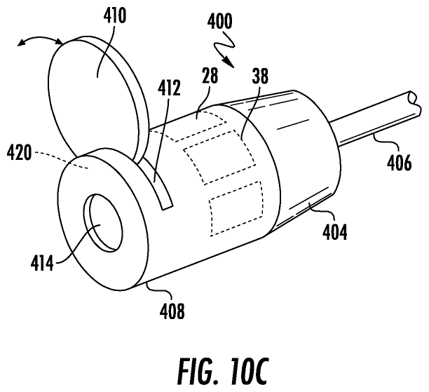

Referring to FIG. 10A-10C, a fifth embodiment of the protected port or catheter is shown generally at 400. The fifth embodiment 400 of the protected port or catheter is shown configured as a catheter with a single tail 402 and cap 404 extending into a single cannula 406, and is configured for intravenous use. The fifth embodiment 400 includes a body 408 surrounding the cap 404 of the catheter 400. The body 408 includes a spring-biased door 410 that slides through a slot 412 on the body 408 between an open position, revealing the cap 404 through an opening 414 on the body 408, and a closed position, hiding the cap 404. The door 410 is spring-biased to the open position. An actuator controls 28 an access control mechanism which may be a latch 416 with a chamfered edge 418 to selectively release the door 410 from the closed position. Activation of the actuator 28, via the control circuit 38, retracts the latch 416, allowing the door 410 to swing to the open position, via a spring 420. The user presses the door 410 back into the slot 412, reengaging the latch 416 with the door 410. The chamfered edge 418e on the latch 416 allows the door 410 to slide the latch 416 laterally out of the way. The tail 402 of the catheter extending down a portion or all of the cannula 406 may include a needle-resistant outer sheath, to prevent insertion of a needle into the tail or cannula 406 to circumvent the door 410 over the cap 404. The outer sheath may be integral to the tail 402 and/or cannula 406.

It should be appreciated by one skilled in the art that the various teachings of this disclosure may be combined or recombined in numerous configurations that may or may not be expressly disclosed herein, Such embodiments are intended to fall within the scope of this disclosure as the most basic form of the disclosure provides for any catheter with a controlled port that disrupts access to its downstream lumen and in turn the patient's blood.

Therefore, it can be seen that the present invention provides a unique solution to the problem of controlling access to a port or catheter and preventing unauthorized use thereof. Consequently, medical professionals and facilities may feel more confident in providing out-patient treatment for patients having or suspected of having substance abuse problems, without the added risk of the patient accidentally overdosing through unauthorized use of the port or catheter. As a result, significant savings may be realized by the treating facility, insurance companies and government health programs.

Those skilled in the art will appreciate that the conception, upon which this disclosure is based, may readily be utilized as a basis for designing other products without departing from the spirit and scope of the invention as defined by the appended claims. Therefore, the claims are not to be limited to the specific examples depicted herein. For example, the features of one example disclosed above can be used with the features of another example. Furthermore, various modifications and rearrangements of the parts may be made without departing from the spirit and scope of the underlying inventive concept. For example, the geometric configurations disclosed herein may be altered depending upon the application, as may the material selection for the components. Thus, the details of these components as set forth in the above-described examples, should not limit the scope of the claims.

What is claimed is:

1. A protected port or catheter, comprising:
a cannula having at least one lumen;
at least one access point in fluid communication with the at least one lumen of the cannula; and
an access control mechanism positioned between the access point and the at least one lumen, wherein said access control mechanism selectively controls fluid communication between the access point and the at least one lumen.

2. The protected port or catheter of claim 1, where the access control mechanism is operable between a closed position and an open position.

3. The protected port or catheter of claim 1, further comprising an actuator configured to operate the access control mechanism between an open position and a closed position.

4. The protected port or catheter of claim 3, further comprising a control circuit configured to operate the actuator.

5. The protected port or catheter of claim 4, further comprising a power source configured to power the control circuit and actuator.

6. The protected port or catheter of claim 1, wherein the access control mechanism is at least one valve.

7. The protected port or catheter of claim 1, where the access control mechanism is at least one door.

8. The protected port or catheter of claim 1, wherein the access control mechanism comprises at least one lock and at least one cover, wherein at least one cover prevents access to the at least one access point.

9. The protected port or catheter of claim 1, wherein the access point comprises a cap with a connector for receiving a syringe or intravenous line.

10. The protected port of catheter of claim 1, wherein the access point comprises a reservoir and a septum.

11. A protected port or catheter, comprising:
a cannula having at least one lumen;
a reservoir at a proximal end of said cannula, the reservoir in fluid communication with the lumen, the reservoir having an access port therein and a septum covering the access port; and
an access control mechanism proximate the septum, wherein the access control mechanism selectively controls access to the reservoir via the septum.

12. The protected port or catheter of claim 11, where the access control mechanism comprises a door.

13. The protected port or catheter of claim 11, further comprising an actuator configured to operate the access control mechanism to permit or prevent access to the septum.

14. The protected port or catheter of claim 13, further comprising:
a control circuit configured to operate the actuator; and
a power source configured to power the control circuit and actuator.

15. The protected port or catheter of claim 11, wherein the access control mechanism is at least one valve in fluid communication between the access port and the at least one lumen of the cannula.

16. A protected port or catheter, comprising:
a cannula having at least one lumen;
at least one cap having an access point therein, the access point in fluid communication with the at least one lumen of the cannula; and
an access control mechanism proximate the access point, wherein the access control mechanism selectively controls access to the access point.

17. The protected port or catheter of claim 16, where the access control mechanism comprises a door.

18. The protected port or catheter of claim 16, wherein the access control mechanism is at least one valve in fluid communication between the at least one access point and the at least one lumen of the cannula.

19. The protected port or catheter of claim 16, wherein the access control mechanism comprises at least one lock and at least one cover, wherein the at least one cover engages the at least one access point.

20. The protected port or catheter of claim 16, further comprising:
an actuator configured to operate the access control mechanism between the open position and the closed position;
a control circuit configured to operate the actuator; and
a power source configured to power the control circuit and actuator.
